(19) United States
(12) Patent Application Publication
(10) Pub. No.: US 2009/0030057 A1
(43) Pub. Date: Jan. 29, 2009

(54) Pharmaceutical composition of telmisartan

(76) Inventors: Shlomit Wizel, Petah Tiqva (IL); Gershon Kolatkar, Petah Tiqva (IL); Erela Zisman, Rishon Le Zion (IL)

Correspondence Address: KENYON & KENYON LLP, ONE BROADWAY, NEW YORK, NY, 10004, US

(21) Appl. No.: 12/151,818

(22) Filed: May 8, 2008

Related U.S. Application Data
(63) Continuation-in-part of application No. 11/286,017, filed on Nov. 22, 2005.
(60) Provisional application No. 60/928,595, filed on May 9, 2007.
(60) Provisional application No. 60/931,559, filed on May 23, 2007.

(51) Int. Cl. A61K 31/4184; C07D 403/10

(52) U.S. Cl. 514/394; 548/305.4

(57) ABSTRACT

The present invention provides a pharmaceutical composition of telmisartan comprising a) a telmisartan compound, b) a surfactant, c) a basic agent, and d) at least one diluent wherein the composition comprises less than 25% of water soluble diluents. The telmisartan compound is preferably present from about 12.5% to about 15.5%. At least one water insoluble diluent is preferred for use in the pharmaceutical composition in an amount from about 40% to about 70% of the total weight of the pharmaceutical composition. A preferred water insoluble diluent is microcrystalline cellulose. Also described are a pharmaceutical composition comprising telmisartan having a powder X-ray diffraction pattern, methods of preparing such pharmaceutical composition, and a crystalline form of Telmisartan.

### CROSS REFERENCE TO RELATED APPLICATIONS

The present application is a Continuation-in-Part application of U.S. application Ser. No. 11/286,017 filed Nov. 22, 2005 and claims the benefit of the following U.S. Provisional Patent Application No. 60/928,595, filed May 9, 2007; and 60/931,559, filed May 23, 2007. The contents of these applications are incorporated herein by reference

### FIELD OF THE INVENTION

The present invention is directed to pharmaceutical compositions of telmisartan having less than 25% of water soluble diluents. The present invention is also directed to a pharmaceutical compositions of crystalline telmisartan, pharmaceutical compositions having a defined powder X-ray diffraction pattern, a polymorphic crystal structure of telmisartan and to processes for the preparation thereof.

### BACKGROUND OF THE INVENTION

Telmisartan is the common chemical name for the compound 4′-[2-n-propyl-4-methyl-6-(1-methylbenzimid-azol-2-yl)benzimidazol-1-ylmethyl]biphenyl-2-carboxylic acid. (CAS Registry No. 144701-48-4.) The empirical formula of telmisartan is C₃₃H₃₀N₄O₂ and its molecular weight is 514.63. The molecular structure of telmisartan is represented by Formula I.

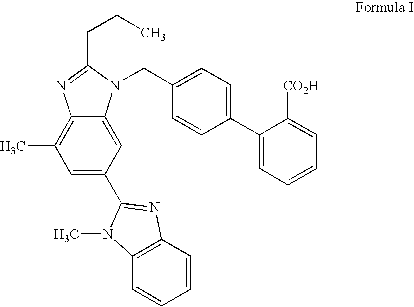

Telmisartan is a non-peptide angiotensin II receptor (type AT₁) antagonist. The United States Food and Drug Administration (FDA) approved it for the treatment of hypertension. It may be used alone or in combination with other hypertensive agents, such as hydrochlorothiazide. Telmisartan is marketed under the trade name Micardis® (telmisartan), available as 20, 40 and 80 mg tablets for oral administration. Two patents are listed in the FDA's electronic Orange Book for telmisartan, U.S. Pat. No. 6,358,986 (“the '986 patent”) and U.S. Pat. No. 5,591,762 (“the '762 patent”).

The '986 patent describes that telmisartan and physiologically acceptable salts thereof can also be used to treat cardiac insufficiency, ischaemic peripheral circulatory disorders, myocardial ischaemia (angina), diabetic neuropathy, glaucoma, gastrointestinal diseases, bladder diseases, and to prevent progression of cardiac insufficiency after myocardial infarct. The '986 patent also describes a mixture of polymorphic forms of telmisartan designated forms A and B (having an endothermic maximum of 269±2° C. and 183±2° C. respectively). Dinnebier et al., Structural Characterization of Three Crystalline Modifications of Telmisartan by Single Crystal and High-Resolution X-ray Powder Diffraction, J. Pharm. Sci. Vol. 89, No. 11, pg 1465-1479 (2000) describes a solvated polymorph C, which is described as having the endothermic effects of polymorph B and two additional small endothermic effects at about 100° C. and 150° C.

In addition to the above therapeutic applications of telmisartan, the '762 patent describes other therapeutic applications, including treating pulmonary diseases, e.g., lung oedema and chronic bronchitis. It also describes using telmisartan to prevent arterial restenosis after angioplasty, thickening of blood vessel walls after vascular operations, and diabetic angiopathy. The '762 patent further describes using telmisartan to alleviate central nervous system disorders, such as depression, Alzheimer's disease, Parkinson Syndrome, bulimia, and disorders of cognitive function in view of the effects of angiotensin on the release of acetylchloine and dopamine in the brain.

Telmisartan is a white to off-white, odorless crystalline powder. It is practically insoluble in water or an aqueous solution in the pH range of 3 to 9, and sparingly soluble in a strong acid, with the exception of hydrochloric acid in which it is insoluble. Telmisartan is soluble in a strong base.

U.S. Pat. No. 6,737,432 describes a crystalline sodium salt of telmisartan, characterized by corresponding x-ray powder diffraction and melting point. A crystalline sodium salt of telmisartan is also described in US 2004/0162327 and US 2006/0276526 which are continuing applications from U.S. Pat. No. 6,737,432. In addition, US 2006/0293377 describes amorphous and crystalline forms of telmisartan sodium. Further, US 2005/0004193 describes pharmaceutical compositions comprising a telmisartan sodium salt or a crystalline form thereof.

In general, Telmisartan is manufactured and supplied in its free acid form. However, as described in WO 00/43370, crystalline Telmisartan apparently exists in two polymorphic forms which have different melting points. Under the influence of heat and humidity polymorph B of Telmisartan, having the lower melting point, reportedly irreversibly transforms into polymorph A, which has a higher melting point. Both of these forms are apparently very poorly soluble in aqueous systems at the physiological pH range in the gastro-intestinal tract of between pH 1 to 7. Further, US 2006/0276525 describes processes for preparing highly pure telmisartan and pharmaceutical compositions thereof.

The published U.S. patent application 2004/0110813 A1, describes that the solubility of Telmisartan can be increased several hundred fold in a pharmaceutical composition comprising 3 to 50% of Telmisartan dispersed in a dissolving matrix comprising a) a basic agent wherein the molar ratio of basic agent: Telmisartan equals 1:1 to 10:1, b) a surfactant or emulsifier in an amount of about 2 to 3% of the final composition, c) 25 to 70% of a water soluble diluent, and optionally 0 to 20% of further excipients and/or adjuvants.

The present invention is directed to pharmaceutical compositions comprising telmisartan in which the solubility of telmisartan is improved, and wherein the composition comprises less than 25% of water soluble diluents.

The invention also relates to the solid state physical properties of telmisartan. These properties may be influenced by controlling the conditions under which telmisartan is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These and other practical physical characteristics are influenced by the conformation and orientation of molecules in the unit cell, which defines a particular form of a substance. Many pharmaceutical solids can exist in different physical forms. Polymorphism is often characterized as the ability of a drug substance to exist as two or more crystalline phases that have different arrangements and/or conformations of the molecules in the crystalline lattice. There is a need in the art for a polymorphic forms of telmisartan and/or processes for preparation thereof.

### SUMMARY OF THE INVENTION

The present invention provides a pharmaceutical composition comprising a) a telmisartan compound, b) a surfactant, c) a basic agent, and d) at least one diluent selected from water soluble and water insoluble diluents, wherein the pharmaceutical composition comprises less than 25% by total weight of the composition of water soluble diluents. Preferably the pharmaceutical composition comprises at least one diluent which is water insoluble, preferably microcrystalline cellulose.

It was determined that, contrary to expextations, telmisartan containing pharmaceutical compositions of the present invention provide sufficient solubility of telmisartan for use in a physiological environment while the amount of water soluble diluents is less than 25% of the composition by weight. Telmisartan is released from said pharmaceutical compositions with sufficient solubility for gastro-intestinal absorption in the slightly acidic and neutral pH region. In one embodiment of the invention dissolution of the pharmaceutical composition in an aqueous solution of neutral pH dissolves at least 80% of the Telmisartan contained therein within 45 minutes. Preferably, at least 80% of the Telmisartan is dissolved from the pharmaceutical composition in such aqueous solution within 30 minutes, and most preferably at least 80% of the Telmisartan is dissolved from the pharmaceutical composition in such aqueous solution within 20 minutes.

In another embodiment of the present invention there is provided a pharmaceutical composition comprising about 12.5% to about 15.5% weight percent of telmisartan; about 40% to about 70% weight percent microcrystalline cellulose; about 2.0% to about 3.5% weight percent of a surfactant, preferably Poloxamer 188; about 9% to about 12% weight percent of a basic agent, preferably Meglumine; about 1.0% to about 1.5% weight percent of a binder; about 7.5% to about 10% weight of a disintegrant; about 0% to 17% weight percent of a water soluble filler and about 0.5% to about 1% weight of a lubricant, all weight percentages are based upon the total weight of the pharmaceutical composition.

Further there is provided a method of preparing a pharmaceutical composition comprising the following steps of 1) mixing a disintegrant, preferably sodium starch glycolate, and one or more diluents, preferably at least one water insoluble diluent, more preferably wherein at least one diluent is microcrystalline cellulose, in a high shear mixer to form a homogeneous mixture; 2) preparing a granulation suspension of purified water, alcohol, a basic agent, a surfactant, and telmisartan; 3) combining the homogeneous mixture and the granulation suspension to form a combined mixture; 4) preparing a granulation solution of water and a binder, preferably Povidone (preferably PVP K-30); 5) adding the granulation solution to the combined mixture to form a granulate; 6) drying the formed granules; 7) sizing the dried granules; 8) mixing the dried granulate with a disintegrant, preferably sodium starch glycolate, and optionally a filler, preferably sorbitol, and; 9) adding a lubricant, preferably magnesium stearate; and 10) compressing the granules into tablets, wherein the prepared pharmaceutical composition comprises less than 25% by weight of water soluble diluents. Further, when the pharmaceutical composition to be prepared is a capsule as opposed to tablets, step 10 of the method is replaced by a step of filling capsules.

In another embodiment the present invention provides a pharmaceutical granulate of telmisartan comprising, at least one water insoluble diluent, a basic agent, a surfactant, and a binder, wherein the amount of water insoluble diluents in the pharmaceutical granulate is about 40% to about 70% by weight of the pharmaceutical granulate, and wherein the pharmaceutical granulate has a powder X-ray diffraction pattern with peaks at about 7.0, 10.8, 13.4, 15.7, 16.9 and 18.1 degrees two-theta ±0.2 degrees two-theta. Preferably, the granulate comprises one or more diluents wherein the amount of water soluble diluents in the pharmaceutical granulate is less than 25% by weight of the pharmaceutical granulate.

In yet another embodiment the present invention provides a pharmaceutical composition in the form of a tablet comprising telmisartan, at least one water insoluble diluent, a basic agent, a surfactant, a binder, a disintegrant, a lubricant and optionally a filler, wherein the amount of water insoluble diluents in the pharmaceutical composition is about 40% to about 70% by weight of the pharmaceutical composition, and wherein the pharmaceutical composition has a powder X-ray diffraction pattern with peaks at about 7.0, 10.8, 13.4, 15.7, 16.9 and 18.1 degrees two-theta ±0.2 degrees two-theta. Preferably, the pharmaceutical composition comprises one or more diluents wherein the amount of water soluble diluents in the pharmaceutical composition is less than 25% by weight of the pharmaceutical composition.

In another embodiment the present invention provides a pharmaceutical composition comprising telmisartan, at least one water insoluble diluent, a basic agent, a surfactant, a binder, a disintegrant, a lubricant and optionally a filler, wherein the pharmaceutical composition is prepared by a process comprising: a) mixing telmisartan with water, a C₁₋₄ lower alkyl alcohol, a basic agent, and a surfactant to obtain a granulation suspension; b) mixing the granulation suspension with a homogeneous mixture of one or more diluents and a disintegrant to obtain a mixture; c) adding a granulation solution comprising water and a binder to the mixture to form a granulate; d) drying the granulate to obtain a granulate, e) sizing the dried granules; f) mixing the dried granulate with a disintegrant, and optionally a filler, g) adding a lubricant to form a final mixture, and h) compressing the final mixture into tablets, wherein the amount of the at least one water insoluble diluent in the pharmaceutical composition is about 40% to about 70% by weight of the pharmaceutical composition and the resulting pharmaceutical composition has a powder X-ray diffraction pattern with peaks at about 7.0, 10.8, 13.4, 15.7, 16.9 and 18.1 degrees two-theta ±0.2 degrees two-theta. Preferably, the pharmaceutical composition comprises one or more diluents wherein the amount of water soluble diluents in the pharmaceutical granulate is less than 25% by weight of the pharmaceutical composition.

In yet another embodiment, the present invention provides a crystalline telmisartan, characterized by X-ray powder diffraction peaks at about 7.0, 10.8, 13.4, 15.7, 16.9 and 18.1 degrees two-theta ±0.2 degrees two-theta.

In another embodiment of the present invention there is provided a method of treating a patient suffering from hypertension comprising administering an effective amount of a telmisartan compound in a pharmaceutical composition comprising about 12.5% to about 15.5% of a telmisartan compound; about 2.0 to about 3.5% of a surfactant; about 9% to about 12% of a basic agent; and at least one diluent, wherein the pharmaceutical composition comprises less than 25% of water soluble diluents. Preferably, the pharmaceutical composition comprises about 40% to about 70% of a water insoluble diluent.

### BRIEF DESCRIPTION OF THE FIGURES

FIG. 1 illustrates the X-Ray Powder Diffraction pattern taken of a telmisartan formulation comprising telmisartan, and having PXRD peaks at 7.0, 10.8, 13.4, 15.7, 16.9 and 18.1 degrees two-theta ±0.2 degrees two-theta.

### DETAILED DESCRIPTION OF THE INVENTION

As used throughout this specification, the term water soluble diluent refers to a compound, used in the art as a diluent, for use in a pharmaceutical composition which is soluble in an aqueous environment. In contrast, the term water insoluble diluent refers to a compound, used in the art as a diluent, for use in a pharmaceutical composition which is insoluble or very poorly soluble in an aqueous environment.

The pharmaceutical composition according to the invention comprises:

a) a telmisartan compound, in admixture with

b) a surfactant,

c) a basic agent, and

d) at least one diluent selected from water soluble and water insoluble diluents, wherein the amount of water soluble diluents in the pharmaceutical composition is less than 25% by weight of the pharmaceutical composition. Preferably, the amount of water soluble diluents in the pharmaceutical composition is less than 20%, more preferably less than 17.5%, by weight of the pharmaceutical composition. Said pharmaceutical composition preferably comprises at least one water insoluble diluent.

Preferably, a pharmaceutical composition of the invention is in tablet dosage form containing telmisartan as the active ingredient in an amount of 20, 40 or 80 mg in each tablet, and wherein the total weight of the pharmaceutical composition is from about 130 mg to about 160 mg. More preferably, such tablets contain 20 mg of telmisartan. The relative amount of the telmisartan compound typically varies from about 12.5% to about 15.5% by weight of the pharmaceutical composition. Further, telmisartan as used in the pharmaceutical composition of the present invention may be in any suitable form such as for example in the form of polymorph A or B.

Suitable diluents for use in the pharmaceutical composition of the invention include microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, potassium chloride, powdered cellulose, sodium chloride, sorbitol and talc. However, the total amount of water soluble diluents in the pharmaceutical composition is less than 25% by weight of the pharmaceutical composition. In a preferred embodiment of the invention at least one diluent for use in the pharmaceutical composition is a water insoluble diluent such as microcrystalline cellulose. Preferably, the insoluble diluent for use in the pharmaceutical composition of the invention is microcrystalline cellulose (e.g. Avicel®PH 102). In such preferred embodiment of the invention, the relative amount of the water insoluble diluent is preferably from about 40% to about 70% by weight of the pharmaceutical composition.

Suitable surfactants for use in the pharmaceutical composition of the invention include poloxamers, polyethylene glycols, polysorbates, sodium lauryl sulfate, polyethoxylated castor oil, and hydroxylated castor oil. A preferred surfactant for use in the pharmaceutical composition of the invention is Poloxamer 188. Preferably, the pharmaceutical composition comprises an amount of about 2.0% to about 3.5% by total weight of the composition of the surfactant.

Suitable basic agents for use in the pharmaceutical composition of the invention include alkaline hydroxides, alkaline carbonates, alkaline phosphates, basic amino acids, and Meglumine. A preferred basic agent for use in the pharmaceutical composition of the invention is Meglumine. Preferably, the pharmaceutical composition comprises an amount of about 9% to about 12% by total weight of the composition of the basic agent.

The telmisartan pharmaceutical composition of the invention may also include pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include fillers, binders, lubricants, etc. Such excipients could be used in the dosage forms of this invention.

Fillers, are added in order to increase the mass of an individual dose to a size suitable for tablet compression. Suitable fillers include for example powdered sugar, calcium phosphate, calcium sulfate, microcrystalline cellulose, lactose, mannitol, kaolin, sodium chloride, dry starch, sorbitol, and talc. A water soluble filler may optionally be used. A preferred filler of this type for use in the pharmaceutical composition of the invention is sorbitol in an amount not exceeding 25% by total weight of the composition. Preferably, the pharmaceutical composition comprises an amount of about 0% to about 17% by total weight of the composition of the optional filler.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydroxyethyl cellulose, hydroxypropyl cellulose (e.g. Klucel®), hydroxypropyl methyl cellulose (e.g. Methocel®), liquid glucose, maltodextrin, methylcellulose, polymethacrylates, povidone (e.g. Povidone PVP K-30, Kollidon®, Plasdone®), pregelatinized starch, sodium alginate and starch. A preferred binder for use in the pharmaceutical composition of the present invention is Povidone (e.g. PVP K-30). Preferably, the pharmaceutical composition comprises an amount of about 1.0% to about 1.5% by total weight of the composition of the binder.

A compacted solid pharmaceutical composition may also include the addition of a disintegrant to the composition. Disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch. A preferred disintegrant for use in the pharmaceutical composition of the invention is sodium starch glycolate. Preferably, the pharmaceutical composition comprises an amount of about 7.5% to about 10% by total weight of the composition of the disintegrant.

Glidants can be added to improve the flowability of a non compacted solid composition and to improve the accuracy of dosing. Excipients that may function as glidants include colloidal silicon dioxide, magnesium trisilicate, powdered cellulose, and talc.

A lubricant can be added to the composition to reduce adhesion and/or ease the release of the product from e.g. the dye. Lubricants include magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate. A preferred lubricant for use in the pharmaceutical composition of the invention is magnesium stearate. Preferably, the pharmaceutical composition comprises an amount of about 0.5% to about 1.0% by total weight of the composition of the lubricant.

Other excipients that may be incorporated into the formulation include preservatives, antioxidants, or any other excipient commonly used in the pharmaceutical industry.

The solid compositions of the present invention include powders, granulates, aggregates and compacted compositions. The dosages include dosages suitable for oral, buccal, and rectal administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, sachets, troches and losenges.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Particularly preferred pharmaceutical compositions of the invention are described in Table 1 infra, wherein all weight percentages are based upon the total weight of the composition.

TABLE 1
Preferred Pharmaceutical Compositions of the Invention

| | | (%) of |
|---|---|---|
| Ingredient | Component | composition |
| Telmisartan | Active Drug | 12.5-15.5 |
| Microcrystalline Cellulose (Avicel PH | Water Insoluble | 40-70 |
| Poloxamer 188 | Surfactant | 2.0-3.5 |
| Meglumine | Basic Agent | 9-12 |
| Povidone (PVP K-30) | Binder | 1.0-1.5 |
| Sodium starch glycolate | Disintegrant | 7.5-10 |
| Sorbitol | Filler | 0-17 |
| Magnesium Stearate | Lubricant | 0.5-1.0 |

The solubility of telmisartan is sufficient for gastro-intestinal absorption from such pharmaceutical compositions of telmisartan in the slightly acidic and neutral pH region. Telmisartan dissolves from the pharmaceutical composition of the present invention at a suitable rate. Preferably, at least 80% of the telmisartan in the pharmaceutical composition dissolves in a neutral aqueous environment within 45 minutes. More preferably, at least 80% of the telmisartan in the pharmaceutical composition dissolves in a neutral aqueous environment within 30 minutes, and most preferably at least 80% of the Telmisartan is dissolved from the pharmaceutical composition in such aqueous solution within 20 minutes.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet. The granulates and powder blends produced by these methods can alternatively be filled into capsules or sachets for example.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

A preferred method of preparing a telmisartan pharmaceutical composition of the invention comprises the steps of 1) mixing a disintegrant, preferably sodium starch glycolate, and one or more diluents, preferably at least one water insoluble diluent, more preferably wherein at least one diluent is microcrystalline cellulose, in a high shear mixer to form a homogeneous mixture; 2) preparing a granulation suspension of purified water, alcohol, a basic agent, a surfactant, and telmisartan; 3) combining the homogeneous mixture and the granulation suspension to form a combined mixture; 4) preparing a granulation solution of water and a binder, preferably Povidone (e.g. PVP K-30); 5) adding the granulation solution to the combined mixture to form a granulate; 6) drying the formed granules; 7) sizing the dried granules; 8) mixing the dried granulate with a filler, preferably sorbitol, and a disintegrant, preferably sodium starch glycolate; 9) adding a lubricant, preferably magnesium stearate to form a final mixture; and 10) compressing the final mixture into tablets, wherein the prepared pharmaceutical composition comprises less than 25% by weight of water soluble diluents. In preparing capsules step 10 will typically be replaced by the step of filling capsule shells. The method of the present invention is however not limited to these mixing procedures or their order, and several of the steps involved can be combined into a single step.

In the above method the amount of disintegrant used preferably results in a pharmaceutical composition comprising about 7.5% to about 10% of a disintegrant. Also, the amount of a telmisartan compound used in said method preferably results in a pharmaceutical composition comprising about 12.5% to about 15.5% of a telmisartan compound. The amount of a basic agent used in said method preferably results in a pharmaceutical composition comprising about 9% to about 12% of a basic agent. The amount of a binder used in said method preferably results in a pharmaceutical composition comprising about 1.0% to about 1.5% of a binder. Further, the amount of surfactant used in said method preferably results in a pharmaceutical composition comprising about 2.0% to about 3.5% of a surfactant. The amount of an optional filler used in said method preferably results in a pharmaceutical composition comprising about 0% to about 17% of a filler. The amount of a lubricant used in said method is preferably from about 0.5% to about 1.0%.

The pharmaceutical composition of the present invention may be a pharmaceutical granulate comprising telmisartan, one or more diluents of which at least one diluent is a water insoluble diluent in an amount of about 40% to about 70% by weight, a basic agent, a surfactant, and a binder, preferably the amount of water soluble diluents in the pharmaceutical granulate is less than 25% by weight of the pharmaceutical granulate, wherein the pharmaceutical composition has a powder X-ray diffraction pattern with peaks at about 7.0, 10.8, 13.4, 15.7, 16.9 and 18.1 degrees two-theta ±0.2 degrees two-theta.

The pharmaceutical composition of the present invention may comprise a telmisartan, one or more diluents of which at least one diluent is a water insoluble diluent in an amount of about 40% to about 70% by weight, a basic agent, a surfactant, and a binder, preferably the amount of water soluble diluents in the pharmaceutical composition is less than 25% by weight of the pharmaceutical composition, wherein the pharmaceutical composition has a powder X-ray diffraction pattern with peaks at about 7.0, 10.8, 13.4, 15.7, 16.9 and 18.1 degrees two-theta ±0.2 degrees two-theta. Preferably, the above pharmaceutical composition further comprises a disintegrant, a lubricant and optionally a filler, and is preferably in the form of a tablet.

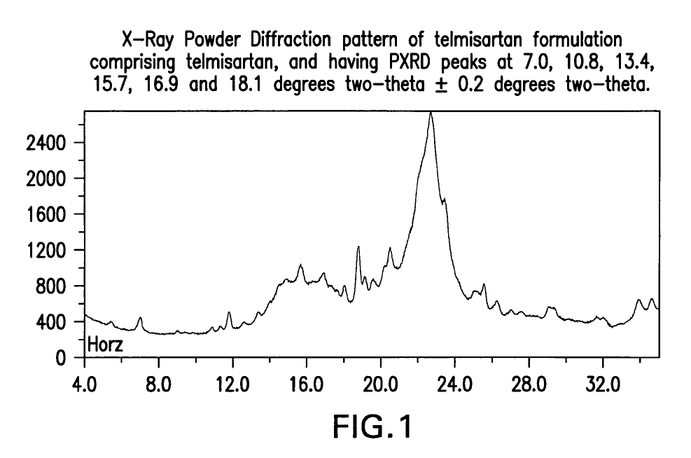

The powder X-ray diffraction pattern of said pharmaceutical composition preferably further contains peaks at about 9.0 and 12.6 degrees two-theta ±0.2 degrees two-theta. A powder X-ray diffraction pattern of the tablet may be substantially as depicted in FIG. 1.

Another embodiment of the present invention provides a pharmaceutical composition comprising a granulate of telmisartan and one or more diluents of which at least one diluent is a water insoluble diluent, in an amount of about 40% to about 70% by weight, wherein the amount of water soluble diluents in the pharmaceutical composition is less than 25% by weight of the pharmaceutical composition prepared by a process comprising: granulating telmisartan with one or more diluents of which at least one diluent is a water insoluble diluent to obtain a granulate comprising telmisartan, wherein the pharmaceutical composition has an X-ray powder diffraction pattern with peaks at about 7.0, 10.8, 13.4, 15.7, 16.9 and 18.1 degrees two-theta ±0.2 degrees two-theta. Preferably, in granulating telmisartan with at least one water insoluble diluent the ratio of telemisartan to the at least one water insoluble diluent is from about 1:4 to about 1:6. In said process granulation can be performed by; mixing telmisartan with water, a C₁₋₄ lower alkyl alcohol, a basic agent, and a surfactant to obtain a granulation suspension; mixing the granulation suspension with a homogeneous mixture of one or more diluents of which at least one diluent is a water insoluble diluent and a disintegrant to obtain a mixture; adding a granulation solution comprising water and a binder to the mixture to form a granulate; and drying the granulate to obtain a granulate comprising telmisartan. Preferably, said pharmaceutical composition is prepared in the form of a tablet. The tablet may further have a powder X-ray diffraction pattern substantially as depicted in FIG. 1.

The above process preferably comprises: 1) mixing a disintegrant, preferably sodium starch glycolate, and one or more diluents, at least one of which is a water insoluble diluent, more preferably wherein the at least one diluent is microcrystalline cellulose, in a high shear mixer to form a homogeneous mixture; 2) preparing a granulation suspension of purified water, a lower alkyl C₁₋₄ alcohol, preferably ethanol, a basic agent, a surfactant, and telmisartan; 3) combining the homogeneous mixture and the granulation suspension to form a combined mixture; 4) preparing a granulation solution of water and a binder, preferably Povidone (preferably PVP K-30); 5) adding the granulation solution to the combined mixture to form a granulate; 6) drying the formed granules; 7) sizing the dried granules; 8) mixing the dried granulate with a disintegrant, preferably sodium starch glycolate, and optionally a filler, preferably sorbitol, and; 9) adding a lubricant, preferably magnesium stearate; and 10) compressing the granules into tablets, wherein the prepared pharmaceutical composition comprises less than 25% by weight of water soluble diluents.

The telmisartan used as a starting material in the process above, may be in the form of polymorph A. Polymorph form A of telmisartan is described in U.S. Pat. No. 6,358,986 (“the '986 patent”), which reference is incorporated herein by reference. Crystalline telmisartan form A may be characterized by an X-ray powder diffraction pattern having peaks at 6.9, 14.4, 15.2, 18.5, 19.1, 20.3, 21.5, 22.5, 24.0 and 25.2±0.2 degrees two theta.

The above pharmaceutical formulations of the present invention comprising a granulate containing telmisartan, such as the one disclosed herein having an X-ray powder diffraction pattern with peaks at about 7.0, 10.8, 13.4, 15.7, 16.9 and 18.1 degrees two-theta ±0.2 degrees two-theta are preferably in a unit dosage form in the form of a tablet. Said pharmaceutical composition of the invention in tablet dosage form containings telmisartan as the active ingredient, preferably in an amount of 20, 40 or 80 mg in each tablet, and wherein the total weight of the pharmaceutical composition is preferably from about 130 mg to about 160 mg. More preferably, such tablets contain 20 mg of telmisartan. The relative amount of the telmisartan compound typically varies from about 12.5% to about 15%.

Suitable diluents for use in the pharmaceutical composition include microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, potassium chloride, powdered cellulose, sodium chloride, sorbitol and talc. However, the total amount of water soluble diluents in the pharmaceutical composition is less than 25%, preferably less than 20%, more preferably less than 15% by weight of the pharmaceutical composition. In the above pharmaceutical compositions of the invention the at least one water insoluble diluent for use in the pharmaceutical composition may be selected from the group consisting of microcrystalline cellulose (e.g. Avicel®), microfine cellulose, lactose, starch, pregelatinized starch, calcium carbonate, calcium sulfate, sugar, dextrates, dibasic calcium phosphate dihydrate, tribasic calcium phosphate, kaolin, magnesium carbonate, magnesium oxide, maltodextrin, powdered cellulose and talc. Preferably, the water insoluble diluent is microcrystalline cellulose, more preferably, the water insoluble diluent for use in the pharmaceutical composition of the invention is microcrystalline cellulose (e.g. Avicel®PH 102). In such preferred embodiment of the invention, the relative amount of the water insoluble diluent is preferably from about 40% to about 70%, more preferably about 50% to about 66%, even more preferably 52% to about 66%, by weight of the pharmaceutical composition.

Suitable surfactants for use in the above pharmaceutical composition of the invention include poloxamers, polyethylene glycols, polysorbates, sodium lauryl sulfate, polyethoxylated castor oil, and hydroxylated castor oil. A preferred surfactant for use in the pharmaceutical composition of the invention is Poloxamer 188. Preferably, the pharmaceutical composition comprises an amount of about 2.0% to about 3.5%, more preferably about 2.0% to about 3.0%, even more preferably about 2.5%, by total weight of the composition of the surfactant.

Suitable basic agents for use in the above pharmaceutical composition of the invention include alkaline hydroxides, alkaline carbonates, alkaline phosphates, basic amino acids, and Meglumine. A preferred basic agent for use in the pharmaceutical composition of the invention is Meglumine. Preferably, the pharmaceutical composition comprises an amount of about 7.5% to about 15%, more preferably about 9% to about 12%, even more preferably of about 9.0% to about 10.0%, by total weight of the composition of the basic agent.

Said telmisartan pharmaceutical composition of the invention may also include pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include fillers, binders, lubricants, etc. Such excipients could be used in the dosage forms of this invention.

Fillers, are added in order to increase the mass of an individual dose to a size suitable for tablet compression. Suitable fillers include for example powdered sugar, calcium phosphate, calcium sulfate, microcrystalline cellulose, lactose, mannitol, kaolin, sodium chloride, dry starch, sorbitol, and talc. A water soluble filler may optionally be used. A preferred filler of this type for use in the pharmaceutical composition of the invention is sorbitol in an amount not exceeding 25% by total weight of the composition. Preferably, the pharmaceutical composition comprises an amount of about 0% to about 17% by total weight of the composition of the optional filler.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydroxyethyl cellulose, hydroxypropyl cellulose (e.g. Klucel®), hydroxypropyl methyl cellulose (e.g. Methocel®), liquid glucose, maltodextrin, methylcellulose, polymethacrylates, povidone (e.g. Povidone PVP K-30, Kollidon®, Plasdone®), pregelatinized starch, sodium alginate and starch. A preferred binder for use in the pharmaceutical composition of the present invention is Povidone (e.g. PVP K-30). Preferably, the pharmaceutical composition comprises an amount of about 0.5% to about 2.5%, more preferably about 1.0% to about 1.5%, by total weight of the composition of the binder.

A compacted solid pharmaceutical composition may also include the addition of a disintegrant to the composition. Disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch. A preferred disintegrant for use in the pharmaceutical composition of the invention is sodium starch glycolate. Preferably, the pharmaceutical composition comprises an amount of about 5.0% to about 10.0%, more preferably about 7.5% to about 10%, by total weight of the composition of the disintegrant.

Glidants can be added to improve the flowability of a non compacted solid composition and to improve the accuracy of dosing. Excipients that may function as glidants include colloidal silicon dioxide, magnesium trisilicate, powdered cellulose, and talc.

A lubricant can be added to the composition to reduce adhesion and/or ease the release of the product from e.g. the dye. Lubricants include magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate. A preferred lubricant for use in the pharmaceutical composition of the invention is magnesium stearate. Preferably, the pharmaceutical composition comprises an amount of about 0.5% to about 2.0%, more preferably of about 0.5% to about 1.0% by total weight of the composition of the lubricant.

Other excipients that may be incorporated into the formulation include preservatives, antioxidants, or any other excipient commonly used in the pharmaceutical industry.

The solid compositions of the present invention include powders, granulates, aggregates and compacted compositions. The dosages include dosages suitable for oral, buccal, and rectal administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

Dosage forms include solid dosage forms like tablets, powders, capsules, sachets, troches and losenges.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Another embodiment of the present invention provides a crystalline form of telmisartan characterized by an X-ray powder diffraction pattern with peaks at about 7.0, 10.8, 13.4, 15.7, 16.9 and 18.1 degrees two-theta ±0.2 degrees two-theta. This crystalline form can be further characterized by X-ray powder diffraction pattern with peaks at 9.0 and 12.6 degrees two-theta ±0.2 degrees two-theta.

The crystalline form of telmisartan in a pharmaceutical composition may be determined may include methods using for example powder X-ray crystallography, Infrared spectroscopy, NMR spectroscopy, thermographic analysis (TGA) and differential scanning calometry (DSC).

Telmisartan is used for the treatment of hypertension in patients, either alone or in combination with other hypertensive agents. The pharmaceutical compositions of the present invention provide an effective delivery system for the administration of telmisartan to patients in need of such treatment. Treatment of hypertensive patients may comprise administering an effective amount of telmisartan in a pharmaceutical composition comprising about 12.5% to about 15% telmisartan, about 2.0 to about 3.5% of a surfactant; about 9% to about 12% of a basic agent; and at least one diluent, wherein the pharmaceutical composition comprises less than 25% of water soluble diluents. Preferably, the pharmaceutical composition comprises about 40% to about 70% of a water insoluble diluent.

Having described the invention with reference to certain preferred embodiments, other embodiments will become apparent to one skilled in the art from consideration of the specification. The invention is further defined by reference to the following example describing in detail the preparation of the compound of the present invention. It will be apparent to those skilled in the art that many modifications, both to materials and methods, may be practiced without departing from the scope of the invention.

### EXAMPLES

### Instruments

### X-Ray Diffraction

XRD was performed using ThermoARL Scintag X-Ray powder diffractometer model X'TRA, Cu-tube, solid state detector. A round standard sample holder with round zero background plate were used. Scanning parameters: Range: 4-35 deg. 2θ: step scan, step size: 0.02 deg., count time: 10 sec.

### Example 1

### Telmisartan Formulations Comprising 41% of a Water Insoluble Diluent

Telmisartan tablets were prepared containing 41% of a water insoluble diluent. Sorbitol, a filler in these formulations, may also be considered a water soluble diluent and is thus present in less than 25%, i.e. 16.7%, of the composition. The tablets were prepared in three potencies, 20, 40 and 80 mg Telmisartan tablets. The tablet compositions are presented in Table 2.

TABLE 2
Telmisartan formulations comprising
41% of a water insoluble diluent.

| | | | | Concen- |
|---|---|---|---|---|
| | 20 mg tablet | 40 mg tablet | 80 mg tablet | tration |
| Ingredients | (mg/tablet) | (mg/tablet) | (mg/tablet) | (% w/w) |
| Telmisartan | 20 | 40 | 80 | 15.4 |
| Microcrystalline | 54 | 108 | 216 | 41.5 |
| Cellulose (Avicel | | | | |
| PH 102) | | | | |
| Poloxamer 188 | 4 | 8 | 16 | 3.1 |
| Meglumine | 15 | 30 | 60 | 11.5 |
| Povidone (PVP | 2 | 4 | 8 | 1.5 |
| K-30) | | | | |
| Sodium starch | 12 | 24 | 48 | 9.2 |
| glycolate | | | | |
| Sorbitol | 21.75 | 43.5 | 87 | 16.7 |
| Magnesium | | | | |
| Stearate | 1.25 | 2.5 | 5 | 1.0 |
| Total | 130.0 | 260.0 | 520.0 | 100.0 |

The tablets were prepared by mixing a homogeneous mixture of microcrystalline cellulose and sodium starch glycolate with a granulation suspension of purified water, Poloxamer 188, Meglumine, and telmisartan in a high shear mixer to form a partial granulate mixture. This partial granulate mixture was granulated with a granulation solution of alcohol and Povidone (PVP K-30). The obtained granules were dried in a Fluid Bed dryer until the observed “loss on drying” (LOD) was 2% or less. The dried granules were then milled by passing them through an oscillating granulator. Subsequently, the milled granulates were mixed with Sorbitol and sodium starch glycolate in a blender. The obtained blend was then mixed with magnesium stearate and tablets were prepared by compressing this mixture.

### Example 2

Telmisartan tablets were prepared containing 53% of a water insoluble diluent. Sorbitol, a filler in these formulations, may also be considered a water soluble diluent and is thus present in an amount less than 25%, i.e. 13.4%, of the composition. The tablets were prepared in three potencies, 20, 40 and 80 mg Telmisartan tablets. The tablet compositions are presented in Table 3.

TABLE 3
Telmisartan formulations comprising
53% of a water insoluble diluent.

| | | | | Concen- |
|---|---|---|---|---|
| | 20 mg tablet | 40 mg tablet | 80 mg tablet | tration |
| Ingredients | (mg/tablet) | (mg/tablet) | (mg/tablet) | (% w/w) |
| Telmisartan | 20 | 40 | 80 | 12.5 |
| Microcrystalline | 84 | 168 | 336 | 52.5 |
| Cellulose (Avicel | | | | |
| PH 102) | | | | |
| Poloxamer 188 | 4 | 8 | 16 | 2.5 |
| Meglumine | 15 | 30 | 60 | 9.375 |
| Povidone (PVP | 2 | 4 | 8 | 1.25 |
| K-30) | | | | |
| Sodium starch | 12 | 24 | 48 | 7.5 |
| glycolate | | | | |
| Sorbitol | 21.4 | 42.8 | 85.6 | 13.375 |
| Magnesium | | | | |
| Stearate | 1.6 | 3.2 | 6.4 | 1.0 |
| Total | 160.0 | 320.0 | 640.0 | 100.0 |

The tablets were prepared by wet granulation by mixing a homogeneous mixture of microcrystalline cellulose and sodium starch glycolate with a granulation suspension of purified water, Poloxamer 188, Meglumine, and telmisartan in a high shear mixer to form a partial granulate mixture. This partial granulate mixture was granulated with a granulation solution of alcohol (ethanol) and Povidone (PVP K-30). The obtained granules were dried in a Fluid Bed dryer until the observed “loss on drying” (LOD) was 2% or less. The dried granules were then milled by passing them through an oscillating granulator. Subsequently, the milled granulates were mixed with Sorbitol and sodium starch glycolate in a blender. The obtained blend was then mixed with magnesium stearate and tablets were prepared by compressing this mixture.

The obtained tablets, when telmisartan form A was used as starting material, were analyzed by XRD and showed to have an X-ray powder diffraction peaks at 7.0, 10.8, 13.4, 15.7, 16.9 and 18.1 degrees two-theta ±0.2 degrees two-theta.

### Example 3

### Telmisartan Formulations Comprising 66% of a Water Insoluble Diluent

Telmisartan tablets were prepared containing 66% of a water insoluble diluent. Moreover, these formulations do not comprise water soluble diluents. The tablets were prepared in three potencies, 20, 40 and 80 mg Telmisartan tablets. The tablet compositions are presented in Table 4.

TABLE 4
Telmisartan formulations comprising
66% of a water insoluble diluent.

| | | | | Concen- |
|---|---|---|---|---|
| | 20 mg tablet | 40 mg tablet | 80 mg tablet | tration |
| Ingredients | (mg/tablet) | (mg/tablet) | (mg/tablet) | (% w/w) |
| Telmisartan | 20 | 40 | 80 | 12.5 |
| Microcrystalline | 105.4 | 210.8 | 421.6 | 65.875 |
| Cellulose (Avicel | | | | |
| PH 102) | | | | |
| Poloxamer 188 | 4 | 8 | 16 | 2.5 |
| Meglumine | 15 | 30 | 60 | 9.375 |
| Povidone (PVP | 2 | 4 | 8 | 1.25 |
| K-30) | | | | |
| Sodium starch | 12 | 24 | 48 | 7.5 |
| glycolate | | | | |
| Magnesium | | | | |
| Stearate | 1.6 | 3.2 | 6.4 | 1.0 |
| Total | 160.0 | 320.0 | 640.0 | 100.0 |

The tablets were prepared by mixing a homogeneous mixture of microcrystalline cellulose and sodium starch glycolate with a granulation suspension of purified water, Poloxamer 188, Meglumine, and telmisartan in a high shear mixer to form a partial granulate mixture. This partial granulate mixture was granulated with a granulation solution of alcohol (ethanol) and Povidone (PVP K-30). The obtained granules were dried in a Fluid Bed dryer until the observed “loss on drying” (LOD) was 2% or less. The dried granules were then milled by passing them through an oscillating granulator. Subsequently, the milled granulates were mixed with Sorbitol and sodium starch glycolate in a blender. The obtained blend was then mixed with magnesium stearate and tablets were prepared by compressing this mixture.

The obtained tablets, when telmisartan form A was used as starting material, were analyzed by XRD and showed to have an X-ray powder diffraction peaks at 7.0, 10.8, 13.4, 15.7, 16.9 and 18.1 degrees two-theta ±0.2 degrees two-theta.

### Example 4

### Preparing a Pharmaceutical Formulation Comprising Telmisartan Having a PXRD Pattern with Peaks at about 7.0, 10.8, 13.4, 15.7, 16.9 and 18.1 Degrees Two-Theta ±0.2 Degrees Two-Theta, from Telmisartan Form A

Tablets were prepared by mixing a homogeneous mixture of microcrystalline cellulose and sodium starch glycolate with a granulation suspension of purified water, Poloxamer 188, Meglumine, and telmisartan form A in a high shear mixer to form a partial granulate mixture. This partial granulate mixture was granulated with a granulation solution of alcohol (ethanol) and Povidone (PVP K-30). The obtained granules were dried in a Fluid Bed dryer until the observed “loss on drying” (LOD) was 2% or less. The dried granules were then milled by passing them through an oscillating granulator. Subsequently, the milled granulates were mixed with Sorbitol and sodium starch glycolate in a blender. The obtained blend was then mixed with magnesium stearate and tablets were prepared by compressing this mixture.

The obtained tablets were analyzed by XRD and showed to have an X-ray powder diffraction peaks at 7.0, 10.8, 13.4, 15.7, 16.9 and 18.1 degrees two-theta ±0.2 degrees two-theta.

### Example 5

### Dissolution Tests with Telmisartan Pharmaceutical Tablet Dosage Forms

Dissolution tests with the Telmisartan pharmaceutical tablet dosage formulations of examples 1 to 3 were performed. In these tests the dissolution of the tablets of examples 1 to 3 was compared with the dissolution of the reference tablet formulation, a Micardis® tablet. These in vitro dissolution tests were conducted using an Apparatus II (Paddle Method) as described in the United State Pharmacopeia XXI/National Formulary XVI. The comparative dissolution tests were conducted under the following conditions. Micardis® tablets and Telmisartan tablets of examples 1, 2, and 3 were dissolved in a USP type II apparatus at a paddle speed of 75 rpm, at a temperature of 37° C., in 900 ml of a buffer at pH 7.5. The results of these dissolution tests are presented in Table 5.

TABLE 5
Comparative dissolution.

| Time | % Dissolution | | | |
|---|---|---|---|---|
| (minutes) | Micardis ® | Example 1 | Example 2 | Example 3 |
| 10 | 53 | 53 | 61 | 38 |
| 20 | 81 | 89 | 99 | 68 |
| 30 | 96 | 99 | 101 | 99 |
| 45 | 100 | 99 | 101 | 99 |
| 60 | 100 | 100 | 102 | 101 |

Based upon these dissolution studies, the results appearing in Table 5 above, it can be concluded that the pharmaceutical tablet formulations of the present invention and the reference Micardis® tablet all dissolve at least 80% of the telmisartan contained therein well within the desired 45 minutes. In fact, all dissolve at least 80% of the telmisartan contained in the formulation within 30 minutes, and some formulations even achieve that at least 80% of the telmisartan from the pharmaceutical composition is dissolved in such aqueous solution within 20 minutes. The results further demonstrate that the pharmaceutical tablet formulations of the invention have a similar dissolution profile compared to the reference formulation.

What is claimed is:

1. A pharmaceutical granulate of telmisartan comprising, at least one water insoluble diluent, a basic agent, a surfactant, and a binder, wherein the amount of water insoluble diluents in the pharmaceutical granulate is about 40% to about 70% by weight of the pharmaceutical granulate, and wherein the pharmaceutical granulate has a powder X-ray diffraction pattern with peaks at about 7.0, 10.8, 13.4, 15.7, 16.9 and 18.1 degrees two-theta ±0.2 degrees two-theta.

2. The pharmaceutical granulate according to claim 1, wherein the pharmaceutical granulate is in the form of a pharmaceutical composition further comprising a disintegrant, a lubricant and optionally a filler.

3. The pharmaceutical granulate according to claim 1, wherein the granulate comprises one or more diluents and wherein the amount of water soluble diluents in the pharmaceutical granulate is less than 25% by weight of the pharmaceutical granulate.

4. The pharmaceutical granulate according to claim 1, wherein the powder X-ray diffraction pattern further comprises peaks at about 9.0 and 12.6 degrees two-theta ±0.2 degrees two-theta.

5. The pharmaceutical granulate according to claim 4, wherein the powder X-ray diffraction pattern is substantially as depicted in FIG. 1.

6. The pharmaceutical granulate according to claim 1, wherein the amount of crystalline telmisartan in the composition is in an amount from about 12.5% to about 15% of the total weight of the composition.

7. The pharmaceutical granulate according to claim 1, wherein the ratio of crystalline telmisartan to the at least one water insoluble diluent is from about 1:4 to about 1:6.

8. The pharmaceutical granulate according to claim 1, wherein the water insoluble diluent is selected from the group consisting of microcrystalline cellulose (e.g. Avicel®), microfine cellulose, lactose, starch, pregelatinized starch, calcium carbonate, calcium sulfate, sugar, dextrates, dibasic calcium phosphate dihydrate, tribasic calcium phosphate, kaolin, magnesium carbonate, magnesium oxide, maltodextrin, powdered cellulose and talc.

9. The pharmaceutical granulate according to claim 8, wherein the water insoluble diluent is microcrystalline cellulose.

10. The pharmaceutical granulate according to claim 1, comprising about 12.5% to about 15% telmisartan, about 2.0% to about 3.5% of the surfactant, about 9% to about 12% of the basic agent, and about 1.0% to about 1.5% of the binder by weight of the total composition.

11. The pharmaceutical granulate according to claim 10, wherein the basic agent is Meglumine, the surfactant is Polyxamer 188, and the binder is Povidone.

12. A pharmaceutical composition in the form of a tablet comprising telmisartan, at least one water insoluble diluent, a basic agent, a surfactant, a binder, a disintegrant, a lubricant and optionally a filler, wherein the amount of water insoluble diluents in the pharmaceutical composition is about 40% to about 70% by weight of the pharmaceutical composition, and wherein the pharmaceutical composition has a powder X-ray diffraction pattern with peaks at about 7.0, 10.8, 13.4, 15.7, 16.9 and 18.1 degrees two-theta ±0.2 degrees two-theta.

13. The pharmaceutical composition according to claim 12, wherein the composition comprises one or more diluents and wherein the amount of water soluble diluents in the pharmaceutical composition is less than 25% by weight of the pharmaceutical composition.

14. The pharmaceutical composition according to claim 12, wherein the powder X-ray diffraction pattern further comprises peaks at about 9.0 and 12.6 degrees two-theta ±0.2 degrees two-theta.

15. The pharmaceutical composition according to claim 14, wherein the powder X-ray diffraction pattern is substantially as depicted in FIG. 1.

16. The pharmaceutical composition according to claim 12, wherein the amount of crystalline telmisartan in the composition is in an amount from about 12.5% to about 15% of the total weight of the composition.

17. The pharmaceutical composition according to claim 12, wherein the ratio of crystalline telmisartan to the at least one water insoluble diluent is from about 1:4 to about 1:6.

18. The pharmaceutical composition according to claim 12, wherein the water insoluble diluent is selected from the group consisting of microcrystalline cellulose (e.g. Avicel®), microfine cellulose, lactose, starch, pregelatinized starch, calcium carbonate, calcium sulfate, sugar, dextrates, dibasic calcium phosphate dihydrate, tribasic calcium phosphate, kaolin, magnesium carbonate, magnesium oxide, maltodextrin, powdered cellulose and talc.

19. The pharmaceutical composition according to claim 18, wherein the water insoluble diluent is microcrystalline cellulose.

20. The pharmaceutical composition according to claim 12, comprising about 12.5% to about 15% telmisartan, about 2.0% to about 3.5% of the surfactant, about 9% to about 12% of the basic agent, about 1.0% to about 1.5% of the binder, about 7.5% to about 10% of the disintegrant, about 0% to about 17% of the water soluble filler, and about 0.5% to about 1.0% of the lubricant by weight of the total composition.

21. The pharmaceutical composition according to claim 20, wherein the basic agent is Meglumine, the surfactant is Polyxamer 188, the disintegrant is sodium starch glycolate, the binder is Povidone, the filler is sorbitol, and the lubricant is magnesium stearate.

22. The pharmaceutical composition of claim 12, wherein the tablet comprises telmisartan in an amount of about 20, 40 or 80 mg in each tablet, and wherein the total weight of the pharmaceutical composition is from about 130 mg to about 160 mg.

23. The pharmaceutical composition of claim 22, wherein the amount of crystalline telmisartan is about 20 mg of telmisartan.

24. A pharmaceutical composition comprising telmisartan, at least one water insoluble diluent, a basic agent, a surfactant, a binder, a disintegrant, a lubricant and optionally a filler, wherein the pharmaceutical composition is prepared by a process comprising: a) mixing telmisartan with water, a C₁₋₄ lower alkyl alcohol, a basic agent, and a surfactant to obtain a granulation suspension; b) mixing the granulation suspension with a homogeneous mixture of one or more diluents and a disintegrant to obtain a mixture; c) adding a granulation solution comprising water and a binder to the mixture to form a granulate; d) drying the granulate to obtain a granulate, e) sizing the dried granules; f) mixing the dried granulate with a disintegrant, and optionally a filler, g) adding a lubricant to form a final mixture, and h) compressing the final mixture into tablets, wherein the amount of the at least one water insoluble diluent in the pharmaceutical composition is about 40% to about 70% by weight of the pharmaceutical composition and the resulting pharmaceutical composition has a powder X-ray diffraction pattern with peaks at about 7.0, 10.8, 13.4, 15.7, 16.9 and 18.1 degrees two-theta ±0.2 degrees two-theta.

25. The pharmaceutical composition according to claim 24, wherein the composition comprises one or more diluents and wherein the amount of water soluble diluents in the pharmaceutical composition is less than 25% by weight of the pharmaceutical composition.

26. The pharmaceutical composition according to claim 24, wherein the powder X-ray diffraction pattern further comprises peaks at about 9.0 and 12.6 degrees two-theta ±0.2 degrees two-theta.

27. The pharmaceutical composition according to claim 26, wherein the powder X-ray diffraction pattern is substantially as depicted in FIG. 1.

28. The pharmaceutical composition according to claim 24, wherein step a) of the process the telmisartan is crystalline telmisartan form A.

29. The pharmaceutical composition according to claim 28, wherein the ratio of telmisartan to the at least one water insoluble diluent is from about 1:4 to about 1:6.

30. The pharmaceutical composition according to claim 24, wherein the amount of crystalline telmisaran in the composition is in an amount from about 12.5% to about 15% of the total weight of the composition.

31. The pharmaceutical composition according to claim 24, wherein the water insoluble diluent is selected from the group consisting of microcrystalline cellulose (e.g. Avicel®), microfine cellulose, lactose, starch, pregelatinized starch, calcium carbonate, calcium sulfate, sugar, dextrates, dibasic calcium phosphate dihydrate, tribasic calcium phosphate, kaolin, magnesium carbonate, magnesium oxide, maltodextrin, powdered cellulose and talc.

32. The pharmaceutical composition according to claim 31, wherein the water insoluble diluent is microcrystalline cellulose.

33. The pharmaceutical composition according to claim 24, comprising about 12.5% to about 15% crystalline telmisartan, about 2.0% to about 3.5% of the surfactant, about 9% to about 12% of the basic agent, about 1.0% to about 1.5% of the binder, about 7.5% to about 10% of the disintegrant, about 0% to about 17% of the water soluble filler, and about 0.5% to about 1.0% of the lubricant by weight of the total composition.

34. The pharmaceutical composition according to claim 33, wherein the basic agent is Meglumine, the surfactant is Polyxamer 188, the disintegrant is sodium starch glycolate, the binder is Povidone, the filler is sorbitol, and the lubricant is magnesium stearate.

35. The pharmaceutical composition of claim 24, wherein the tablet comprises crystalline telmisartan in an amount of about 20, 40 or 80 mg in each tablet, and wherein the total weight of the pharmaceutical composition is from about 130 mg to about 160 mg.

36. The pharmaceutical composition of claim 35, wherein the amount of crystalline telmisartan is about 20 mg of telmisartan.

37. Crystalline telmisartan, characterized by an X-ray powder diffraction pattern with peaks at about 7.0, 10.8, 13.4, 15.7, 16.9 and 18.1 degrees two-theta ±0.2 degrees two-theta.

38. The crystalline telmisartan of claim 37, further characterized by an X-ray powder diffraction pattern with peaks at 9.0 and 12.6 degrees two-theta ±0.2 degrees two-theta.
